(19) United States
(12) Patent Application Publication
(10) Pub. No.: US 2022/0096635 A1
(43) Pub. Date: Mar. 31, 2022

(54) Liposomal Nanoparticle

(71) Applicant: NewSouth Innovations Pty Limited, Sydney (AU)

(72) Inventors: Ewa Goldys, Sydney (AU); Wei Deng, Sydney (AU); Yagiz Alp Aksory, Sydney (AU)

(21) Appl. No.: 17/427,648

(22) PCT Filed: Jan. 31, 2020
(86) PCT No.: PCT/AU2020/050067

§ 371 Date: Aug. 1, 2021

(30) Foreign Application Priority Data
Jan. 31, 2019 (AU) 2019900286

(51) Int. Cl. A61K 41/00; C12N 15/11; A61K 47/69; C12N 9/22

(52) U.S. Cl. CPC A61K 41/0028; A61K 41/0071; C12N 15/111; C12N 2320/32; C12N 9/22; C12N 2310/20; A61K 47/6911

(57) ABSTRACT

The invention relates to a liposomal nanoparticle comprising: a liposomal vehicle comprising: one or more liposome forming lipids; and one or more destabilising agents capable of forming reactive oxygen species when exposed to an inducer; and a genome editing agent, or part thereof, and compositions and kits comprising the liposomal nanoparticle.

### FIELD OF THE INVENTION

The present invention relates to liposomal nanoparticles for delivery of genome editing agents, to compositions comprising liposomal nanoparticles for delivery of genome editing agents, to methods of producing liposomal nanoparticles for delivery of genome editing agents, to methods for spatiotemporal control over genome editing, and to methods of editing a genome of a cell.

### BACKGROUND

Genome editing is a technique that allows researchers to directly manipulate a host genome, aiding in the production of animal models and cell lines for disease and biological studies and believed to be beneficial in therapeutic applications. Direct genomic manipulation of a host genome is achieved by inserting, replacing or removing DNA from a genome by utilising engineered nucleases. The engineered nucleases create double-strand breaks (DSBs) and single-strand breaks (SSBs) at specific locations in the DNA and harness the naturally occurring DNA repair mechanisms to selectively alter the host genome. During DNA repair, either the non-homologous end joining (NHEJ) or homology directed recombination (HDR) pathways are used to repair the host DNA which, depending on the manipulation result in mutations effective for gene disruptions and knockouts, deletions and insertions.

An example of a genome editing tool is based on a bacterial CRISPR (clustered regularly interspaced short palindromic repeats)-associated protein-9 nuclease (Cas9) from a bacterium, S. thermophiles. This CRISPR/Cas9 system is an adaptive immune response system present in some prokaryotic cells, in which the Cas9 endonuclease is used by the CRISPR system to recognise and destroy foreign DNA entering into the cell (Barrangou, R., et al., Science, 2007. 315(5819): p. 1709-1712; Jinek, M., et al., Science, 2012: p. 1225829). The applications of this system have been extended to various fields, including biological research (Shen, B., et al., Cell research, 2013. 23(5): p. 720), human medicine (Veres, A., et al., Cell stem cell, 2014. 15(1): p. 27-30), biotechnology (Sampson, T. R. and D. S. Weiss, Bioessays, 2014. 36(1): p. 34-38) and agriculture (Khatodia, S., et al., Frontiers in plant science, 2016. 7: p. 506).

Despite the great promise of the genome-editing systems such as CRISPR-Cas9, several challenges remain to be addressed before its successful application for human patients. While there are still challenges to the nascent genome editing techniques, safe and efficient delivery of the system to target cells in vivo remains to be one of the major challenges.

Improved approaches for controlled delivery of the genome editing systems to target cells and tissue represent an unmet need.

### SUMMARY

The inventors have found that genome editing by genome editing agents can be controlled by delivering the genome editing agents to cells in liposomal vehicles into which have been incorporated one or more destabilising agents. The destabilising agent forms reactive oxygen species when exposed to an inducer, resulting in destabilisation of the liposome and release of the genome editing agent from the liposome. The release of the genome editing agent is therefore controllable by controlling exposure of the liposome to the inducer.

A first aspect provides a liposomal nanoparticle for delivery of a genome editing agent, or part thereof, comprising:
(a) a liposomal vehicle comprising:
   (i) one or more liposome forming lipids; and
   (ii) one or more destabilising agents capable of forming reactive oxygen species when exposed to an inducer; and
(b) a genome editing agent, or part thereof.

A second aspect provides a composition comprising a liposomal nanoparticle for delivery of a genome editing agent or part thereof, the liposomal nanoparticle comprising:
(a) a liposomal vehicle comprising:
   (i) one or more liposome forming lipids; and
   (ii) one or more destabilising agents capable of forming reactive oxygen species when exposed to an inducer; and
(b) a genome editing agent, or part thereof.

A third aspect provides a liposomal system for delivery of a genome editing agent or part thereof, comprising:
(a) a liposomal vehicle comprising:
   (i) one or more liposome forming lipids; and
   (ii) one or more destabilising agents capable of forming reactive oxygen species when exposed to an inducer; and
(b) a genome editing agent, or part thereof;
   wherein the genome editing agent or part thereof is released from the liposome by exposure to the inducer.

A fourth aspect provides a liposomal nanoparticle for delivery of a CRISPR complex or part thereof, comprising:
(a) a liposomal vehicle comprising:
   (i) one or more liposome forming lipids; and
   (ii) one or more destabilising agents capable of forming reactive oxygen species when exposed to an inducer; and
(b) a CRISPR complex or part thereof.

A fifth aspect provides a composition comprising a liposomal nanoparticle for delivery of a CRISPR complex or part thereof, the liposomal nanoparticle comprising:
(a) a liposomal vehicle comprising:
   (i) one or more liposome forming lipids; and
   (ii) one or more destabilising agents capable of forming reactive oxygen species when exposed to an inducer; and
(b) a CRISPR complex or part thereof.

A sixth aspect provides a liposomal system for delivery of a CRISPR complex or part thereof, comprising:
(a) a liposomal vehicle comprising:
   (i) one or more liposome forming lipids; and
   (ii) one or more destabilising agents capable of forming reactive oxygen species when exposed to an inducer; and
(b) a CRISPR complex or part thereof;
   wherein the CRISPR complex or part thereof is released from the liposomal vehicle by exposure to radiation.

A seventh aspect provides a method of modifying a genome of a cell, comprising administering the liposomal nanoparticle of the first aspect, or the composition of the second aspect, to a cell, and exposing the liposomal nanoparticle to an inducer to thereby destabilise the liposome and release the genome editing agent.

An eighth aspect provides a method of modifying a genome of a cell, comprising administering the liposomal nanoparticle of the fourth aspect, or the composition of the fifth aspect, to a cell, and exposing the liposomal nanoparticle to an inducer to thereby destabilise the liposome and release the CRISPR complex or part thereof.

A ninth aspect provides a method of preparing a liposomal nanoparticle, comprising combining one or more liposome forming lipids, one or more destabilisers that are capable of forming reactive oxygen species when exposed to an inducer, and a genome editing agent or part thereof, under conditions which promote formation of a liposomal vehicle encapsulating the genome editing agent or part thereof.

A tenth aspect provides a method of preparing a liposomal nanoparticle, comprising combining one or more liposome forming lipids, one or more destabilisers that are capable of forming reactive oxygen species when exposed to an inducer, and a CRISPR complex or part thereof, under conditions which promote formation of a liposomal vehicle encapsulating the CRISPR complex or part thereof.

An eleventh aspect provides a kit for preparing a liposomal nanoparticle of the first aspect, comprising:
(i) one or more liposome forming lipids;
(ii) one or more destabilising agents capable of forming reactive oxygen species when exposed to an inducer; and
(iii) optionally, a genome editing agent, or part thereof.

A twelfth aspect provides a kit for preparing a liposomal nanoparticle of the fourth aspect, comprising:
(i) one or more liposome forming lipids;
(ii) one or more destabilising agents capable of forming reactive oxygen species when exposed to an inducer; and
(iii) optionally, a CRISPR complex or part thereof.

A thirteenth aspect provides a liposomal vehicle for preparing a liposomal nanoparticle of the first or fourth aspect, the liposomal vehicle comprising:
(i) one or more liposome forming lipids; and
(ii) one or more destabilising agents capable of forming reactive oxygen species when exposed to an inducer.

A fourteenth aspect provides a method of controlling genome editing, comprising:
(a) administering the liposomal nanoparticle of the first or fourth aspect; and
(b) directing the inducer to a location where release of the genome editing agent is desired.

A fifteen aspect provides a method of controlling genome editing, comprising:
(a) administering the liposomal nanoparticle of the first or fourth aspect; and
(b) directing electromagnetic radiation of at least 100 eV to a location where release of the genome editing agent is desired.

### BRIEF DESCRIPTION OF THE FIGURES

FIGS. 1A and B is TEM images of examples of representative liposome samples incorporating verteporin as described herein. Scale bar is 500 nm.

FIG. 2 is graphs (A and B) showing characterization of liposomes incorporating verteporfin. (A) is a graph showing size distribution of liposome suspension. (B) is a graph showing absorption and fluorescence spectra of verteporfin loaded inside liposomes. Arrows indicate the characterized peaks of verteporfin.

FIG. 3 is graphs showing (A) size and distribution of liposome suspensions in DI water with or without light illumination (2, 4 and 6 minutes); and (B) VP release profile from the intact liposome samples under light illumination (2, 4 and 6 minutes), without light, and following chemical disruption.

FIG. 4 A. is confocal images and quantitative analysis of GFP expression level in HEK293 cells at 48 hr after the different treatment conditions as indicated. The concentration of the liposomes was 50 μg/mL. Scale bars=30 μm. The box is bounded by the first and third quartile with a horizontal line at the median and whiskers extend to 1.5 times the interquartile range. The mean value was analysed using the t test (n=4). ***, p<0.001, compared to the liposome group without light. B. is a western blot showing GFP and b-Actin expression following the indicated treatment of HEK293 cells.

FIG. 5 is an image showing spatial control of GFP fluorescence intensity with light-triggered Cas9 sgRNA release from the liposomes. The indicated spot (dashed circle) was irradiated with a 690 nm LED for 4 min and the petri dishes were photographed 48 hr later under IVIS spectrum in vivo imaging system.

FIG. 6 is a schematic illustration of quantitative readout detection system in vivo. (A) shows an overview of the visual knock-out readout in zebrafish. (B) is a schematic representation of zebrafish cross-section showing slow muscles forming a single layer of parallel fibers underneath the zebrafish skin. (C) is a confocal section of smyhc1:eGFP zebrafish line under brightfield and green channel. Scale bars: 75 μm. (D) is a schematic representation of an sgRNA-Cas9 complex targeting the eGFP expression driven by slow muscle-specific smyhc1 promoter.

FIG. 7 is images and qualitative and quantitative assessment of light-triggered release of CRISPR/Cas9 in zebrafish. (A) is fluorescence images of smyhc1-eGFP zebrafish (3dpf); uninjected negative controls, co-injected with Cas9 and liposome/CRISPR complex without light exposure, co-injected with Cas9 and liposome/CRISPR complex with 5 min light exposure, and injected with only CRISPR/Cas9 as positive control; (B) is a graph showing qualitative assessment of the knockout rate in zebrafish images by total fluorescence intensity. (C) is a graph showing quantification of CRISPR/Cas9-mediated knockout rates in zebrafish by number of knocked-out slow-muscle fibers at single cell resolution. Scale bars: 500 μm, main image and 100 μm, partially enlarged images.

FIG. 8 is images and qualitative and quantitative assessment of the effect of light exposure time on controlled release of CRISPR/Cas9. (A) is fluorescence images of smyhc1-eGFP zebrafish (3dpf) co-injected with Cas9 and liposome/CRISPR complex with no light exposure; 1 min; 2 min and 5 min. (B) is a graph showing qualitative assessment of the effects of light exposure times on the efficiency of CRISPR/Cas9-mediated knockout in zebrafish embryos; and (C) is a graph showing quantitative assessment of the effects of light exposure times on the efficiency of CRISPR/Cas9-mediated knockout in zebrafish embryos. Scale bars: 500 μm, main image and 100 μm, partially enlarged images.

FIG. 9 is graphs showing (A) Quantitative assessment of Cas9-mediated knockout by light-triggered release of CRISPR in zebrafish by counting the number of knock-out slow muscle fibers per embryo under different treatment as indicated in the image; and (B) Effect of light exposure time on controlled release of CRISPR/Cas9 by counting the number of knock-out slow muscle fibers per embryo at the different illumination time points.

FIG. 10 is graphs showing assessment of light and liposome toxicity to zebrafish embryos. (A) is a graph showing survival of 3dpf zebrafish injected with different CRISPR/Cas9 to liposome concentrations; and (B) is a graph showing survival rate of zebrafish embryos exposed to different duration of light.

FIG. 11 is graphs showing the survival and knockout rates (%) of zebrafish embryos. (A) is a graph showing survival of zebrafish embryos exposed to different duration of time of light (between 0-60 mins) and (B) is a graph showing percent survival and knockout in zebrafish embryos injected with different CRISPR/Cas9 to liposome ratios (between 1:0-1:5).

FIG. 12 is a graph showing the relative gene expression of TNFAIP3 to GAPDH after different treatment conditions as indicated (from left to right: control; control+TNFγ; commercial liposomes loaded with CRISPR; home-made liposomes loaded CRISPR; home-made liposomes+4 Gy; home-made liposomes loaded CRISPR+4 Gy; 4 Gy alone).

FIG. 13 is: (A) an image of the results of agarose gel electrophoreses of sgRNA (3 μg/mL) and mixture of sgRNA and VP (3 μg/mL sgRNA and 16 μg/mL verteporfin) after light illumination at different time points. From right to left lane: the control, the mixture, 2 min, 4 min and 6 min; and (B) The viability of HEK293 cells after incubation with the liposomes for 2 hr and illumination at 690 nm for 4 min. The concentration of liposomes was 25, 50 and 100 μg/mL. The viabilities are expressed as mean percentages and standard deviation (n=4) relative to control cells.

FIG. 14 is 3D rendered confocal images of: (A) control transgenic smyhc1:eGFP embryos show individual slow-muscle fibers expressing eGFP as a single layer; and (B) transgenic embryos injected with eGFP-targeting CRISPR guide RNA show loss of green fluorescent signal from slow-muscle fibers.

FIG. 15 is the amino acid sequence of an example of a Cas9 protein.

### DETAILED DESCRIPTION

The present disclosure relates to liposomal nanoparticles for delivery of genome editing agents. Genome editing agents are: molecules, or complexes of molecules, which are capable of creating deletions, insertions and/or mutations in DNA of a cell, such as in genes and/or genomes of a cell, in vivo; or DNA which encodes molecules, or complexes of molecules, which are capable of creating deletions, insertions and/or mutations in DNA of a cell, such as in genes and/or genomes of a cell, in vivo. In embodiments where a genome editing agent comprises more than one component, a part of a genome editing agent is a component of the genome editing agent.

An example of a genome editing agent is a CRISPR complex, typically comprised of a CRISPR guide RNA and an endonuclease, such as Cas9 endonuclease. CRISPR complexes are based on a prokaryotic immune system which functions by storing fragments of invading bacteriophage. In subsequent infections of the bacteria by bacteriophage, RNA comprising sequence encoded by the stored bacteriophage DNA, complexed with specialised RNA and cellular nucleases, target and cleave bacteriophage DNA in the subsequent infection. CRISPR complexes combine the cleavage function of the prokaryotic immune system, with sequence which is complementary to a desired nucleotide sequence, to target the cleavage function to the desired nucleotide sequence to thereby cleave the desired sequence. Introduction of a CRISPR complex into tissue or cells allows targeted in vivo genome editing of the cellular genome.

Prior to the present disclosure, genome editing agents have been delivered into cells using viral vectors and conventional liposomes. The use of viral vectors and conventional liposomes to deliver genome editing agents, such as CRISPR complexes, into tissue or cells results in release of the CRISPR in an uncontrollable manner. This can result in genome editing occurring in unintended cells or tissues. As the genome editing capabilities of CRISPR are capable of altering the genome (as opposed to altering mRNA produced from the genome), such unintended genome editing can lead to unacceptable outcomes, particularly in a clinical setting.

The inventors have found that genome editing agents, such as CRISPR complexes or a part thereof, can be effectively delivered to cells in a controlled manner by using a liposome in which an inducible destabilising agent has been incorporated.

Accordingly, in one aspect, there is provided a liposomal nanoparticle for delivery of genome editing agents, comprising:
(a) a liposomal vehicle comprising:
   (iii) one or more liposome forming lipids; and
   (iv) one or more destabilising agents capable of forming reactive oxygen species when exposed to an inducer; and
(b) a genome editing agent, or a part thereof.

In one embodiment, the genome editing agent is a Clustered Regularly Interspaced Short Palindromic repeats (CRISPR) complex or part of a CRISPR complex.

As described herein, liposome nanoparticles encapsulating a CRISPR complex, and comprising verteporfin as a destabilising agent, produce reactive oxygen species on exposure to light radiation. The reactive oxygen species cause oxidation of the liposomal lipids, resulting in destabilisation of the liposome, and release of the CRISPR complex. Thus, incorporating a destabilising agent such as verteporfin into the liposome allows the encapsulated genome editing agent, such as CRISPR complex, to be controllably released from the liposomal vehicle by exposure to an inducer, such as light radiation.

The liposomal nanoparticle described herein therefore lends itself to targeted controlled release of a CRISPR complex payload in vivo using radiation, such as X-ray, gamma-radiation, or light, as an inducer triggering the release of the CRISPR complex at the site of interest. In this regard, the liposome itself need not be targeted to the site of intended CRISPR activity, but rather the inducer (e.g. X-ray radiation, gamma ray radiation or light) targeted to the site to induce release of the CRISPR complex or part thereof.

The liposomal nanoparticles described herein therefore permit targeted release of the genome editing agent in the intended cells or tissues by conjugating the liposome with targeting molecules or just using non-targeted liposomes even if it is not possible to target the liposomal particles themselves to intended cells or tissues. Such an approach would be particularly advantageous in situations where the intended calls or tissues do not have a known targeting moiety, or when targeting cells or tissue in areas which cannot be easily accessed or cannot be easily differentiated from surrounding areas.

The liposomal vehicle of the liposomal nanoparticle comprises one or more liposome forming lipids. A liposomal vehicle is a liposome which is capable of encapsulating an agent. The liposome forming lipids may be any suitable lipids that are capable of forming liposomes. Liposomes are generally formed by the self-assembly of dissolved lipid molecules, each of which contains a hydrophilic head group and hydrophobic tails. These lipids take on associations which yield entropically favourable states of low free energy, in some cases forming bimolecular lipid leaflets. Such leaflets are characterized by hydrophobic hydrocarbon tails facing each other and hydrophilic head groups facing outward to associate with aqueous solution. At this point, the bilayer formation is still energetically unfavourable because the hydrophobic parts of the molecules are still in contact with water, a problem that is overcome through curvature of the forming bilayer membrane upon itself to form a vesicle with closed edges. This free-energy-driven self-assembly is stable and has been exploited as a powerful mechanism for engineering liposomes specifically to the needs of a given system. Lipid molecules used in liposomes are conserved entities with a head group and hydrophobic hydrocarbon tails connected via a backbone linker such as glycerol. Cationic lipids commonly attain a positive charge through one or more amines present in the polar head group. The presence of positively charged amines facilitates binding with anions such as those found in DNA. The liposome thus formed is a result of energetic contributions by Van der Waals forces and electrostatic binding to the DNA which partially dictates liposome shapes. Because of the polyanionic nature of DNA, cationic (and neutral) lipids are typically used for gene delivery, while the use of anionic liposomes has been fairly restricted to the delivery of other therapeutic macromolecules (Balazs and Godbey, 2011).

Examples of cationic lipids include N-[1-(2,3-dioleyloxy)propyl]-N,N,N-trimethylammonium chloride (DOTMA), [1,2-bis(oleoyloxy)-3-(trimethylammonio)propane] (DOTAP), 3β[N—(N′, N′-dimethylaminoethane)-carbamoyl] cholesterol (DC-Chol), and dioctadecylamidoglycylspermine (DOGS). Dioleoylphosphatidylethanolamine (DOPE), a neutral lipid, can be used in conjunction with cationic lipids because of its membrane destabilizing effects at low pH, which aide in endolysosomal escape.

Examples of suitable lipids include 1,2-dioleoyl-sn-glycero-3-phosphocholine (DOPC) and 1, 2-di-(9 Zoctadecenoyl)-3-trimethylammonium-propane (DOTAP), or 1,2-dioleoyl-sn-glycero-3-phosphoethanolamine (DOPE), N-[1-(2,3-dioleyloxy)propyl]-N,N,N-trimethylammonium chloride (DOTMA), Hydrogenated Soy L-α-phosphatidylcholine (HSPC) and 1,2-dioleoyl-sn-glycero-3-phosphoethanolamine-N-(hexanoylamine) (PE-NH₂).

In one embodiment, the one or more liposome forming lipids comprise DOPC and DOTAP.

In some embodiments, the liposomal vehicle further comprises cholesterol.

The liposomal vehicle comprises one or more destabilising agents capable of forming reactive oxygen species when exposed to an inducer. Typically, the one or more destabilising agents are incorporated in the liposome lipid bilayer. As used herein, a “destabilising agent capable of forming reactive oxygen species when exposed to an inducer” is a compound or molecule which, when incorporated into the lipid bilayer of a liposome and exposed to an inducer, produces reactive oxygen species (ROS)(¹0⁻₂). The reactive oxygen species typically cause oxidation of the lipids of the liposome, resulting in destabilisation of the liposome.

In one embodiment, the destabilising agent is an inorganic nanoparticle or a metal nanoparticle. Suitable metal nanoparticles include gold, silver and bismuth that can enhance X-ray or gamma-ray radiation and energy transfer from X-ray or gamma-ray radiation. In one embodiment, the metal nanoparticle is gold nanoparticles.

In one embodiment, the destabilising agent is a photosensitiser. Suitable photosensitizers include verteporfin (VP), rose bengal, aminolevulinic acid, photofrin, 5-aminolevulinic acid and protoporphyrin IX

In one embodiment, the destabilising agent is VP. VP (trade name Visudyne) is a benzoporphyrin derivative that is traditionally used as a photosensitizer for photodynamic therapy to eliminate the abnormal blood vessels in the eye associated with conditions such as the wet form of macular degeneration.

As described herein, when a photosensitiser is used as a destabiliser, visible light or ionising radiation can be used as the inducer.

In some embodiments, the one or more destabilisers is a photosensitizer and the inducer is visible light.

In one embodiment, the one or more destabilisers is a photosensitizer and the inducer is ionising radiation, such as X-ray radiation or gamma ray radiation.

In some embodiments, the one or more destabilisers is a combination of metal nanoparticles and photosensitizers. For example, gold nanoparticles and VP are effective at destabilising liposomes when exposed to X-ray radiation or gamma ray radiation.

The genome editing agent is typically encapsulated by the liposome. In one embodiment, the genome editing agent is a CRISPR complex or part thereof. The CRISPR complex may be any CRISPR complex known in the art. CRISPR complexes for genome editing, including cleavage of DNA, deletion of DNA, insertion of DNA, and mutation of DNA, are known in the art and described in, for example, WO 2014/204729; WO2014/204726; WO 2015/071474; WO 2017/064546. CRISPR complexes and kits for preparing CRISPR complexes are commercially available from, for example, New England Biolabs, Inc.

The CRISPR complex typically comprises a guide RNA and a modifying polypeptide (e.g., CRISPR associated protein). The guide RNA directs the activities of the CRISPR associated protein (Cas) (e.g., a site-directed modifying polypeptide such as Cas9) to a specific desired target sequence within a target DNA. As used herein, a “guide RNA” comprises a DNA-targeting sequence and a protein-binding sequence (e.g., a tracrRNA). Typically, the guide RNA comprises a DNA targeting sequence and tracrRNA. The tracr RNA comprises a sequence which associates with a modifying polypeptide.

The DNA-targeting sequence of the guide RNA comprises a nucleotide sequence that is complementary to a target sequence in a target DNA. The DNA-targeting sequence of the guide RNA hybridises with a target DNA to thereby guide the bound modifying protein into proximity with the target sequence to allow cleavage. Thus, the nucleotide sequence of the DNA-targeting sequence determines the location within the target DNA that the guide RNA and the target DNA will interact. The DNA-targeting sequence of a guide RNA can be modified (e.g., by recombinant DNA techniques) to hybridize to any desired sequence within a target DNA. Typically, the target DNA sequence is adjacent a PAM sequence (NGG).

The DNA-targeting sequence typically has a length of from about 20 nucleotides to about 22 nucleotides. In various embodiments, the DNA-targeting sequence that is complementary to a target sequence of the target DNA is 14, 15, 16, 17 18, 19, 20, 21, 22, 23, 24, 25, 26 or 27 nucleotides in length.

The protein-binding sequence of a guide RNA interacts with a site-directed modifying polypeptide. Typically, the modifying polypeptide is a nuclease, more typically an endonuclease. The guide RNA guides the bound modifying polypeptide to a specific nucleotide sequence within target DNA via the above-mentioned DNA-targeting sequence. The protein-binding sequence of a guide RNA comprises two stretches of nucleotides that are complementary to one another (crRNA and sequence within the tracrRNA). The complementary nucleotides of the protein-binding segment hybridize to form a double stranded RNA duplex (dsRNA), and in embodiments in which the guide RNA is a single molecule, a stem-loop structure. The protein-binding sequence of a guide RNA is about 20 (e.g., 19) nucleotides in length, which is comprised of a short sequence of about 4 nucleotides, and a repeat stem loop of about 12 nucleotides.

In some embodiments, the guide RNA can be formed from two RNA molecules. Typically, the guide RNA is a single molecule (a single guide RNA (sgRNA)).

As noted above, the CRISPR complex comprises a guide RNA and a modifying protein. Typically, the modifying protein is a nuclease, more typically an endonuclease. The nuclease may be any nuclease suitable for use with CRISPR. Typically, the nuclease is a CRISPR associated protein (Cas). The liposome typically comprises a CRISPR associated (Cas) protein, as part of the CRISPR complex. The Cas protein is a modifying protein which is guided to the target by the guide RNA and cleaves the target DNA. Examples of suitable Cas proteins include Cas9 and Cas12a, or variants thereof. In some embodiments, the Cas protein is Cas9 or a variant thereof. Variants of Cas9 are known in the art and are described in, for example, WO 2016/196655. Variants of Cas9 are also commercially available from, for example, New England Biolabs, Inc.

As used herein, a part of a CRISPR complex is a component of a CRISPR complex that separately does not form the complete CRISPR complex. An example of a component of a CRISPR complex is a Cas protein, or a guide RNA. It is envisaged that in some embodiments, different parts of the CRISPR complex can be packaged into separate liposome particles and delivered to the same cell to allow a functional CRISPR complex to form within the cell.

It will be appreciated by those skilled in the art that the genome editing agent within the liposome can be guide RNA and Cas protein (or RNA encoding the Cas protein), or DNA capable of expressing the guide RNA and Cas protein.

In various embodiments, the genome editing agent or part thereof comprises:
(a) a guide RNA;
(b) a Cas protein, typically Cas 9 protein or variant thereof;
(c) a guide RNA and a Cas protein (e.g., Cas9);
(d) a DNA sequence encoding a guide RNA;
(e) a DNA or RNA sequence encoding a Cas protein (e.g., Cas 9);
(f) a DNA sequence encoding a guide RNA and a Cas protein;
(g) a guide RNA and a DNA sequence encoding a Cas protein.

In one embodiment, the genome editing agent or part thereof is a CRISPR complex or part thereof.

In one embodiment, the CRISPR complex or part thereof comprises guide RNA and a Cas protein.

In one embodiment, the CRISPR complex or part thereof comprises Cas protein without the guide RNA.

In one embodiment, the CRISPR complex or part thereof comprises guide RNA without the Cas protein.

Typically, the Cas protein is Cas9, or a variant thereof.

In some embodiments, the CRISPR complex or part thereof may further comprise a cationic polymer. The cationic polymer may be one or more polymers selected from the group consisting of poly-L-lysine, polyamidoamine, poly[2-(N,N-dimethylamino)ethyl methacrylate], chitosan, poly-L-ornithine, cyclodextrin, histone, collagen, dextran, and polyethyleneimine.

The liposome surface may be further modified with targeting material to enable enhanced uptake of the liposomes into a target region or target cells of a subject. The material may be an antigen, antibody, antibody fragment, peptide, hormone, cytokine, ligand and receptor. For example, liposome folate conjugates have been used to make liposomes tumour cell-specific due to folate receptor overexpressed on many cancer cells. The conjugation can be synthesized using methods described in, for example, Gabizon et al, 1999, Bioconjugate chemistry, 1999. 10(2): p. 289-298.

As used herein, an inducer is an agent which causes the destabilising agent to produce reactive oxygen species. In some embodiments, the inducer is radiation. The inducer may be any form of radiation which causes the destabilising agent to produce reactive oxygen species. Typically, the inducer is electromagnetic radiation. It will be appreciated by those skilled in the art that the inducer used with the liposomal nanoparticle will depend on the destabilising agent employed. In embodiments in which the destabilising agent is a photosensitiser, the inducer may be light, or high energy electromagnetic radiation. In embodiments in which the inducer is light, the light may be visible light of UV light. The light typically has a wavelength in the range of from 350 nm to 400 nm and, 400 nm to 800 nm, more typically 600 nm to 800 nm, or 670 nm to 700 nm In some embodiments, the light is of wavelength about 690 nm. In some embodiments, the wavelength is about 405 nm.

In some embodiments, the electromagnetic radiation is ionising radiation. Typically, the ionising radiation has energy greater than 100 eV. Examples of ionising radiation are X-ray radiation or gamma-ray radiation. In some embodiments, typically in which the destabilising agent is a metal nanoparticle, or a metal nanoparticle and a photosensitiser, the electromagnetic radiation is high energy electromagnetic radiation. High energy electromagnetic radiation typically has energy higher than about 5 keV, for example, an energy of about 320 KV., or about 6 MeV. In various embodiments, the high energy electromagnetic radiation is in the range of from about 5 keV to about 7 MeV, about 50 KeV to about 6 MeV, about 100 Key to about 6 MeV, about 200 KeV to about 6 MeV, about 300 KeV to about 6 MeV.

Also provided is a composition comprising the liposomal nanoparticle described herein. In some embodiments, the composition is a pharmaceutical composition comprising the liposomal nanoparticle described herein and a pharmaceutically acceptable carrier. Methods for the formulation of liposomes with pharmaceutical carriers are known in the art and are described in, for example, Goodman & Gillman's: The Pharmacological Basis of Therapeutics (11^{th} Edition, McGraw-Hill Professional, 2005).

Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol, xylitol, erythritol, maltitol or sorbitol; starch, acacia, rubber, alginate, gelatine, calcium phosphate, calcium silicate, cellulose, methyl cellulose, microcrystalline cellulose, polyvinyl pyrrolidone, water, methyhydroxybenzoate, propylhydroxybenzoate, talc, magnesium stearate.

Administration of the agent to a subject may be by intravenous, intraperitoneal, subcutaneous, intramuscular, intranasal or intrathecal injection. Compositions suitable for intravenous, intraperitoneal, subcutaneous, intramuscular, intranasal or intrathecal use include sterile aqueous solutions or dispersions and sterile powders for the extemporaneous preparation of sterile injectable solutions or dispersions. The pharmaceutically acceptable carrier can be a solvent or dispersion medium containing, for example, water, ethanol, polyol (for example, glycerol, propylene glycol, liquid polyethylene glycol and the like), suitable mixtures thereof.

One aspect provides a method of preparing a liposomal nanoparticle, comprising combining one or more liposome forming lipids, one or more destabilisers, and a CRISPR complex or part thereof, under conditions which promote formation of a liposomal vehicle encapsulating the CRISPR complex or part thereof.

In some embodiments, the method comprises combining one or more liposome forming lipids, one or more destabilisers, and optionally cholesterol, in chloroform to form a bilayer film. The film is then disrupted with vigorous mixing with an aqueous liquid such as, for example, HEPES and PBS to form liposomal vehicles. Separately, CRISPR complex or a part thereof, is typically combined with a cationic polymer such as PEI, to form a composite. The composite and liposomal vehicle are incubated to permit incorporation of the composite into the liposomal vehicle. In some embodiments, the composite, in an aqueous liquid, is combined with the liposome film and the liposomal nanoparticles formed following vigorous mixing of the film and composite.

Another aspect provides a method of modifying a genome of a cell, such as modifying a gene and/or gene expression in a cell, comprising administering the liposomal nanoparticle of the first aspect, or the composition of the second aspect, to a cell, and exposing the liposomes to radiation to thereby destabilise the liposome and release the CRISPR complex or part thereof. The gene may be modified in any way that CRISPR is capable of modifying a gene. The genome may be modified by, for example, deletion of DNA sequence, insertion of DNA sequence, or mutation of DNA sequence.

In some embodiments, the cell is in a subject.

As used herein, the term “subject” refers to a mammal such as a human, primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Typically the mammal is a human or primate. More typically, the mammal is a human. Although the present invention is exemplified using a zebrafish model, this is not intended as a limitation on the application of the present invention to that species, and the invention may be applied to other species, in particular, humans.

A further aspect provides a kit for preparing a liposomal nanoparticle of the first aspect, comprising:
(i) one or more liposome forming lipids;
(ii) one or more destabilising agents capable of forming reactive oxygen species; and
(iii) a Clustered Regularly Interspaced Short Palindromic repeats (CRISPR) complex or part thereof.

In one embodiment, the kit comprises a liposomal vehicle comprising one or more liposomes and one or more destabilising agents, and a CRISPR complex or part thereof. In some embodiments, the liposomal vehicle further comprises cholesterol.

As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

In order to exemplify the nature of the present invention such that it may be more clearly understood, the following non-limiting examples are provided.

### EXAMPLES

### Example 1—Spatial and Temporal Control of CRISPR/Cas Gene Editing Via a Light-Triggered Liposome System

Liposome nanoparticles have been studied and widely used in nucleic acid and drug delivery as one of advanced carriers (Ewe, A., et al., Storage stability of optimal liposome-polyethylenimine complexes (lipopolyplexes) for DNA or siRNA delivery. Acta biomaterialia, 2014. 10(6): p. 2663-2673; Majzoub, R. N., et al., Patterned threadlike micelles and DNA-tethered nanoparticles: a structural study of PEGylated cationic liposome—DNA assemblies. Langmuir, 2015. 31(25): p. 7073-7083]. For the traditional liposomes, passive release of the encapsulated cargos was often too slow to achieve an optimal therapeutic effect. This has stimulated new efforts towards the development of new-generation liposomes with activated release, accelerating content release rates and enhancing the therapeutic efficacy (Kono, K., et al., Multifunctional liposomes having target specificity, temperature-triggered release, and near-infrared fluorescence imaging for tumor-specific chemotherapy. Journal of Controlled Release, 2015. 216: p. 69-77; Liu, X., et al., Fusogenic reactive oxygen species triggered charge-reversal vector for effective gene delivery. Advanced Materials, 2016. 28(9): p. 1743-1752; Carter, K. A., et al., Porphyrin—phospholipid liposomes permeabilized by near-infrared light. Nature communications, 2014. 5: p. 3546). External light source is one of promising stimuli employed in activation of liposomes due to adjustable spectrum regions, illumination intensities and times. Furthermore, spatial and temporal control of the light source provides an extra benefit to precisely monitor the cargo release. By taking advantage of the light triggering modality, we have designed the light-triggered liposome formulation where a photosensitive molecule, verteporfin, is incorporated inside a liposomal bilayer (Chen, W., W. Deng, and E. M. Goldys, Light-triggerable liposomes for enhanced endolysosomal escape and gene silencing in PC12 cells. Molecular Therapy-Nucleic Acids, 2017. 7: p. 366-377; WenJie, C., et al., Photoresponsive endosomal escape enhances gene delivery using liposome-polycation-DNA (LPD) nanovector. Journal of Materials Chemistry B, 2018). In addition, incorporating cholesterol (Chol) in the liposomal formulation can improve resistance to liposome aggregation in a physiological environment, protect them from protein binding and mechanical breakage and prolong their half-lives (Yang, S.-y., et al., Comprehensive study of cationic liposomes composed of DC-Chol and cholesterol with different mole ratios for gene transfection. Colloids and Surfaces B: Biointerfaces, 2013. 101: p. 6-13).

Herein we further demonstrate in vivo transfection efficacy of the same liposome formulation by delivering Cas9 protein and gRNA complexes (Cas9 RNPs) into zebrafish embryos and to establish the optimal conditions for transfection. We prepared light-triggered liposome formulation by using the previous method established in our lab. We complexed liposomes with Cas9 RNP and microinjected the mixture solution into embryos expressing eGFP gene, followed by light illumination at 690 nm for 6 min. To establish a simple and quantitative readout for gene knockout we focused on the large slow-muscle cells in the zebrafish trunk. Zebrafish slow-muscle is a single layer of parallel fibers that encase the fish beneath the skin, rendering them accessible to rapid and accurate quantitation by fluorescence microscopy. To test the feasibility of this approach, we used a double transgenic zebrafish strain that expressed eGFP under the control of the slow-muscle smyhc1 promoter. To evaluate the efficiency of the sgRNA, we targeted a region in eGFP and confirmed the loss of eGFP fluorescence in individual slow-muscle cells at 72 hours post-fertilization (hpf).

Materials and Methods:

Lipids (DOTAP and DOPE) were purchased from Avanti Polar Lipids (Alabaster, Ala., USA). Verteporfin, cholesterol (Chol) and chloroform were purchased from Merck Australia. Dulbecco's modified Eagle's medium, fetal bovine serum, trypsin, optiMEM, Dulbecco's Phosphate-buffered saline, Truecut cas9 v2, GFP sgRNA and lipofectamine were purchased from ThermoFisher Australia. Zyppy Plasmid MiniPrep Kit was purchased from Zymo Research. MEGAshortscript T7 kit and mirVana miRNA isolation kit were purchased from Invitrogen Australia. Cas9 protein used in vivo experiments was obtained from Toolgen, Inc.

Liposome Formulation Preparation

The liposome formulation was prepared based on our previous method with minor modification (Deng, W., et al., Nature Communications. 2018. 9(1): p. 2713). Briefly, lipid components of DOTAP, DOPE and Chol at mole ratio of 1:1:1 were mixed with verteporfin (16 μM) in 500 μL chloroform, or DOTAP, DOPE, Chol and verteporfin at a mole ratio of 1:0.94:1:0.06 were mixed with 500 μl chloroform. The mixture solvent was then evaporated under argon gas stream. The thin lipid film was formed around the wall of the test tube and hydrated with HEPES buffer (40 mM, pH 7.4) or DI water by vigorous stirring for 30 min until the suspension was homogenized. The hydrated suspension was left for 2 hours at room temperature to allow the complete hydration of the lipids. The hydrated liposome suspension was extruded 11 times through a 200 nm polycarbonate membrane in a mini-extruder. The resulting suspension was stored at 4° C. under argon. For preparation of liposomes incorporating Cas9 gRNA RNP, the lipid film was fully resuspended in 500 μL DI water solution containing gRNA (0.01 μM) and Cas9 protein (0.1 mg mL⁻¹), followed by the hydration procedure described above.

To determine the encapsulation amount of VP loaded inside of liposomes, we added Triton X-100 (0.1%) to as-prepared liposome solution, resulting in VP release. The VP fluorescence (excitation/emission: 425/690 nm) was recorded on a Fluorolog-Tau-3 system and compared with the corresponding VP standard curve.

Characterization

The zeta potential and size distribution of liposome samples were determined by DLS using a Zetasizer 3000HSA. After 2 min balance at 25° C., each sample was measured in triplicate and data were collected as the mean±standard deviation (SD). Prior to transmission electron microscopy (TEM) imaging of liposome sample, the TEM grid specimens were prepared using the negative staining method. Briefly, a copper grid was placed onto a drop of 10 μL liposome suspension, allowing the grid to absorb samples for 3 min, followed by staining with 2% (w/v) phosphotungstic acid for another 3 min. After air-dry of the sample overnight, the grid specimens were then observed under a TEM (Philips CM 10) with an acceleration voltage of 100 KV. Images were captured with the Olympus Megaview G10 camera and processed with iTEM software.

The absorption and fluorescence spectra of liposomes and pure VP were measured with a UV-VIS spectrometer (Cary 5000, Varian Inc.) and a Fluorolog-Tau3 System (HORIBA Scientific) with 425 nm Xe lamp excitation, respectively.

Assessment of In Vitro VP Release Profile Under Light Illumination and Serum Stability of the Liposomes

100 μL liposome suspension was diluted in PBS (pH 7.4) and activated by LED light illumination (0.15 mW/cm²) at 690 nm for 2 min, 4 min and 6 min. The samples were then dialyzed in D-Tube Dialyzer (Merck Millipore). These devices were kept in 50 mL centrifuge tubes with 12 mL PBS in a shaker (80 rpm) for 24 hours. At various time points (0 hr, 1 hr, 3 hr, 6 hr and 24 hr), an aliquot of PBS was taken for the fluorescence characterisation of the released VP. The total VP fluorescence was measured by disrupting liposomes with 0.1% Triton X-100. The percentage of VP release (Rᵥₚ(%)) at various time points was calculated as follows:

$${R}_{v p} ( \% ) = \frac{{F}_{t} - {F}_{0}}{{F}_{max} - {F}_{0}} \times 100 \% \qquad 1$$

where Fₜ and F₀ respectively indicates the fluorescence intensity of released VP at various time points and without illumination. Fₘₐₓ refers to the total fluorescence intensity of VP after the disruption of liposomes by adding 0.1% Triton X-100.

Cellular Uptake of the Liposomes in HEK293 Cells

A transgenic HEK293 containing GFP gene in the genome (Thermo Fisher via MTA) was used in cell experiments. They were grown in DMEM containing 10% fetal bovine serum and 1% antibiotics. The cells (1×10⁵ cells/well) were attached to glass-bottom petri dishes and incubated at 37° C. for 24 hr. After removing the culture medium, the cells were incubated with liposome suspension (50 μg/ml) in cell medium for 1 hr, 2 hr and 4 hr. The cells were then washed with PBS (1×, PH 7.4) three times to remove free liposomes. To assess the cellular uptake activity of the liposomes, the cells were stained with DRAQ5™ (5 μM, ab108410, Abcam) for 10 min before imaging. The cells were imaged using an Olympus FV3000 confocal laser scanning microscopy system. Laser sources at 405 nm and 640 nm was used for the excitation of VP and DRAQ5™, respectively.

Assessment of In Vitro GFP Gene Transfection Via Light-Triggered Liposomes

Before transfection, HEK293 were seeded on glass-bottom perti dishes at the density of 1×10⁵ cells/well, followed by overnight incubation. Liposome suspension (50 μg/ml) incorporating Cas9 gRNA RPN was added to each well. After 2 hour incubation, the old medium was replaced by the fresh one, followed by illumination of LED light (0.15 mW/cm²) at 690 nm for 2 min, 4 min and 6 min, respectively. After the treatments, the cells were incubated for another 48 hours. The GFP fluorescence signal from the cells was imaged using under a FV3000 confocal laser scanning microscope. A laser at 488 nm was used for GFP excitation. Quantitative analysis of GFP fluorescence intensity from the cells was conducted by using ImageJ software. The GFP knockout efficacy under different experimental conditions are expressed as mean percentages and standard deviation (n=4) relative to control cells without any treatment. For the imaging of spatial control of CRISPR release from the liposomes, the petri dishes were photographed at 48 hr under IVIS Spectrum In Vivo Imaging System (PerkinElme, USA) after the treatments. The excitation/emission wavelength for GFP fluorescence imaging was 465 nm/560 nm. Toxicity assays of liposomes and light on cells and the effect of singlet oxygen on sgRNA

For liposome and light toxicity experiments, HEK293 cells (1-4×10⁴ mL⁻¹) were grown on 96-well plates in a culture medium with 10% FBS for 24 hr, followed by incubation with the liposome suspension for 2 hours and light illumination afterwards. After the treatments, the old medium was removed and a fresh medium was added to cells. At 24 hours, the cytotoxicity of the liposomes and light on the cells was determined by the MTS test (Promega Co., USA) according to manufacturer's instructions and compared with control cells without any treatment. Cell viability was then calculated as a percentage of the absorbance of the untreated control sample. The latter was set to 100%.

For the assessment of singlet oxygen's effect on sgRNA, the mixture solution of sgRNA and verteporfin (3 μg/mL sgRNA and 16 μg/mL verteporfin) was respectively exposed to light illumination at different time points (0, 2, 4 and 6 min). After treatment, 10 uL of each sample was mixed with 2 uL 6× loading dye (Thermo Fisher) for gel electrophoresis on 2.5% agarose gel (Sigma-Aldrich, Australia) with 1×SYBR Gold (Thermo Fisher) loaded. The gel electrophoresis was carried out in 1×TBE buffer (10.8 g of Tris base, 5.5 g of boric acid, 4 ml of 0.5 M EDTA, 1 L D/D water, pH 8.4) at 110v for 50 mins. Gel image was photographed under UV light using Bio-Rad gel Doc XR+ system.

Western Blotting Analysis

After treatments HEK293 cells were washed twice with PBS and lysed with RIPA buffer (Thermo Fisher Scientific) supplemented with a protease inhibitor cocktail (Thermo Fisher Scientific) according to the protocol by the manufacturer. Total protein was extracted and loaded in the wells of Bis-Tris protein gel (Thermo Fisher Scientific). After separation the protein was transferred to PVDF membranes (Thermo Fisher Scientific). The membranes were blocked with BSA blocking buffer (Thermo Fisher Scientific) at 4° C. overnight and incubated with GFP polyclonal antibody (A11122, Life Technologies Australia Pty Ltd, 1:1000 dilution) for 1 hr at room temperature. After washing with TBST three times, the membranes were incubated with corresponding HRP-conjugated secondary antibody (1:1000 dilution) for 1 hr at room temperature. After washing with TBST three times, the membranes were visualized using enhanced chemiluminescence reagents on a ChemiDoc™ MP Imaging System (Bio-Rad Laboratories, Inc., USA).

Zebrafish Embryos

Zebrafish embryos and adults were maintained and handled according to zebrafish facility SOPs, approved by Animal Research Ethics Committee at Macquarie University and in compliance with the Animal Research Act, 1985 and the Animal Research Regulation, 2010. Adult zebrafish were maintained under standard conditions. Acta1:eBFP2; smyhc1:eGFP line was obtained by crossing Tg(acta1:eBFP2)pc5 (Cole et al. 2011, PLoS, 9(10): e1001168) and Tg(smyhc1:eGFP) (Elworthy et al. 2008, Development, 135(12):2115-2126) strains.

Target Site Selection

For the initial screen, CRISPR sgRNA target sites were selected manually within the early 5′ region of eGFP gene that match the sequence GN18GNGG according to Schier et al 2014. To avoid any off-targets, these sites were checked for uniqueness in BLASTN (Zv9) using Bowtie and Bowtie2 methods, and the pre-defined specificity rules that not tolerate any mismatch in the first ten 3′ bases of the target site.

Production of sgRNA

To generate templates for sgRNA transcription, target gene-specific complementary oligonucleotides containing the 20 base target site without the PAM, were annealed to each other, then cloned into a plasmid (px330, Addgene) containing T7 promoter sequence and tracrRNA tail. The resulting sgRNA template was purified using Zyppy Plasmid MiniPrep Kit (Zymo Research).

For making CRISPR sgRNA, the template DNA (from the step above) was first linearized by BamHI digestion, then purified using a QIAprep column. Crispr sgRNA is generated by in vitro transcription using MEGAshortscript T7 kit (Invitrogen). After in vitro transcription, the sgRNA (˜140 nucleotides long) was purified using mirVana miRNA isolation kit (Invitrogen). The size and quality of resulting sgRNA was confirmed by electrophoresis through a 3% (wt/vol) low-range agarose gel. Recombinant Cas9 protein was obtained from Toolgen, Inc.

Microinjection of Liposome-Cas9 RNPs into Zebrafish Embryos

On the day of injections, the injection mix was prepared as follows:

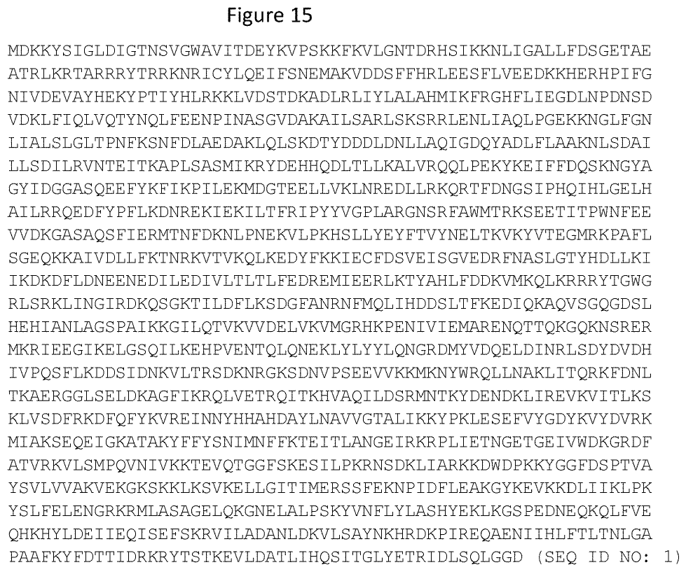

TABLE 1

| | | | Injection MasterMix | |
|---|---|---|---|---|
| | Injection MasterMix | | (Control) | |
| | | [Final] | | [Final] |
| Contents | 1x (μl) | (ng/μl) | 1x (μl) | (ng/μl) |
| sgRNA | 3.5 | 75 | 3.5 | 75 |
| Cas9 Protein (TrueCut | 1.0 | 500 | 1.0 | 500 |
| Cas9 Protein v2) (SEQ | | | | |
| ID NO: 1) (FIG. 15) | | | | |
| Liposome | 2.5 | n/a | — | — |
| Phenol Red | 1.0 | n/a | 1.0 | n/a |
| Water | — | — | 2.5 | 0 |

For the initial screen, zebrafish TAB WT embryos were collected. Injection components were mixed and incubated at room temperature for 5 mins to form complex, then stored on ice. The injection mastermix was loaded into the needle and microinjected into zygotes using standard zebrafish injection protocols. Delivery of 2n1 of injection mixture into the single cell (not the yolk) aimed. The injected eggs were grown in 1× egg water in 100 mm plastic petri dish and kept in the incubator at 28° C. Embryo density did not exceed more than 60 embryos in 25 mL egg water per petri dish. Some uninjected embryos (control group) were kept from the same clutch and grown at 28° C. Embryos were grown to 48-72hpf.

In Vivo Recombination Analysis

Embryos with developmental defects were sorted out at the end of 24hpf, 48hpf and 72hpf. Only morphologically normal looking embryos were kept. Approximately 70-80% of embryos appear normal at 72hpf. At 72hpf, 16 embryos were randomly selected and anesthetised using Tricane. Anesthetised fish were mounted on 1% low-melting agarose in glass bottomed 35 mm Petri dishes. The trunk of mounted embryos was screened for eGFP signal using Leica DMi3000 inverted microscope.

Genotyping

High Resolution Melting (HRM) analysis was used for rapid and efficient identification of CRISPR-Cas9 induced somatic mutations. HRM is a fluorescence based assay which measures the amount of dsDNA at different temperatures, thus revealing the melting temperature (Tm) of a PCR product of interest. While a homoduplex product generated from a homozygous DNA sample will have a particular Tm, a heteroduplex product generated from a heterozygous individual will have an additional Tm, generally a much lower Tm signature. It is this heteroduplex signature that expedites identification of mutant alleles. To identify the somatic mutagenesis in CRISPR-Cas9 injected embryos, genomic DNA (gDNA) was extracted from pools of 8 embryos using the HotSHOT method. The resulting gDNA was analysed using HRM assay. The successful hits from HRM assay are further genotyped by polymerase chain reaction (PCR).

Microscopy

Images were taken on a Leica DMi3000 inverted microscope and Zeiss confocal microscope. Fish embryos were embedded on 1% low-melting agarose in 35 mm glass bottom petri dish. Sections were focus-stacked using Zerene Stacker software. Virtual cross section of the fish embryos was generated and analysed using Imarls software.

Results:

Characterization of Liposomes

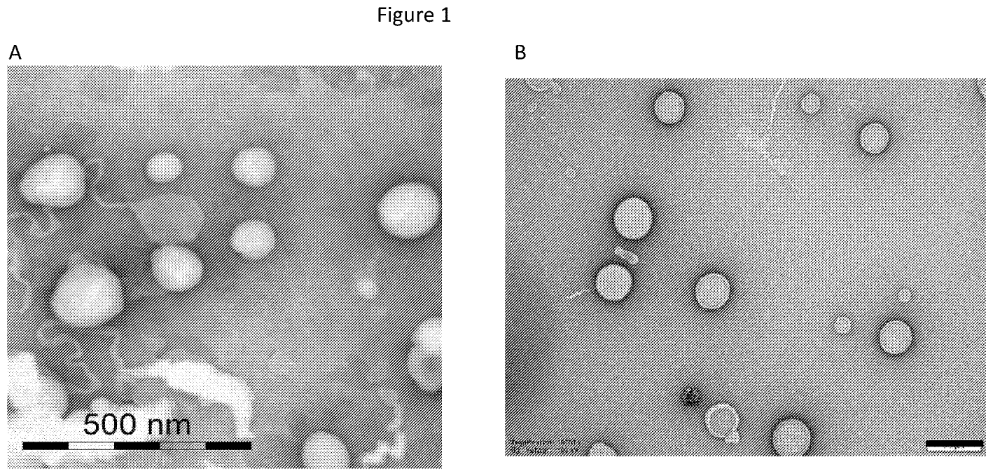

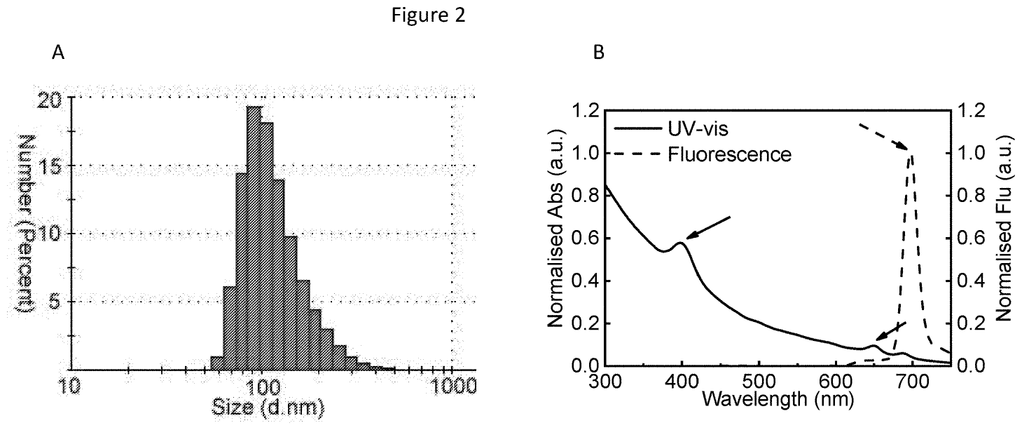

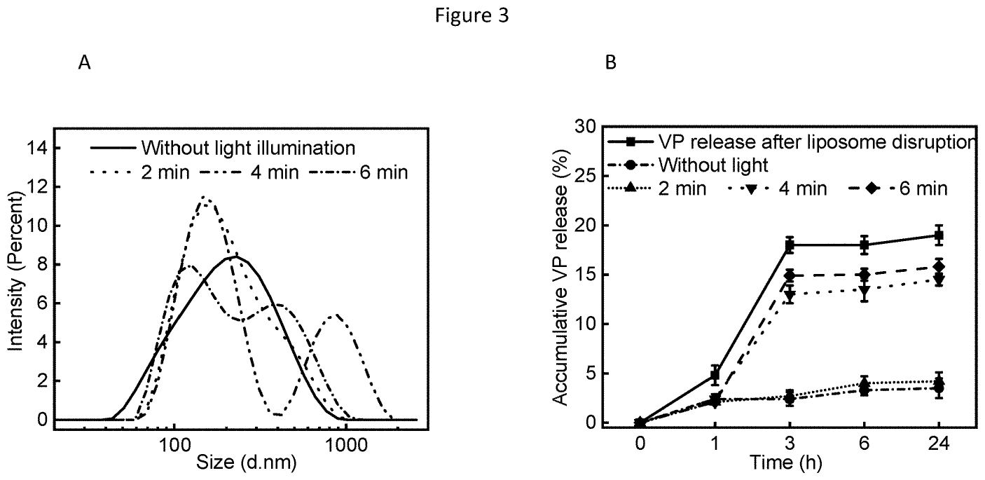

FIG. 1 shows the typical TEM images of liposomes loaded with verteporfin. The average size was about 167.5+/−1.9 nm. The size distribution and zeta potential of liposomes (FIG. 2A and FIG. 3A) were confirmed by dynamic light scattering. The surface charge was determined to be 28+/−1.1 mV.

The absorption and fluorescence spectra of verteporfin loaded inside liposomes were demonstrated in FIG. 2B, where the characterised peaks of VP were clearly observed, as indicated in the FIG. 2B. The structure disruption and releasing behaviors of the prepared liposomes after light illumination were respectively investigated in DLS experiments and VP release measurements. FIG. 3B shows the size distribution of liposome solutions without and with light illumination. The mean size of the liposomes was reduced after 2 min illumination, compared to the liposomes without light illumination. However the size distribution of these two samples was similar (0.4386 v.s. 0.4573). The well-separated peaks of the liposomes appeared after 4 min and 6 min, with the first individual peak representing the smaller size of the liposomes and the second indicating that of liposome aggregates. The results revealed that after light illumination at 4 min and 6 min, most of the liposomes were damaged into small pieces with some pieces forming aggregates. FIG. 3B shows the proportion of VP release from the liposome samples with and without light illumination. The similar VP release profile was observed between the liposomes with light triggering and under 2 min illumination, indicating 2 min illumination did not obviously affect the liposome structure. By contrast, the release of VP accelerated markedly at 3 hr after 4 min and 6 min light illumination, with cumulative release reaching the similar amount of VP released from the liposomes totally disrupted by Triton X-100. The findings indicated that enough amount of ROS was generated from VP with longer illumination time (4 min and above in this study), oxidising unsaturated the lipid bilayer and inducing the VP release from the liposomes.

Assessment of Cellular Uptake Activity of the Liposomes and In Vitro GFP Gene Knockout

In order to investigate the cellular uptake of liposomes, HEK293 cells were treated with the liposomes for 1 hr, 2 hr and 4 hr. Higher red fluorescence signal from VP was observed after 2 hr incubation than the cells treated for 1 hr. After 4 hr incubation, the red signal from VP was not significantly changed compared with 2 hr incubation period. However, some clusters were also observed in other regions due to non-specific binding (data not shown). Therefore, we chose 2 hr incubation time for HEK293 cells.

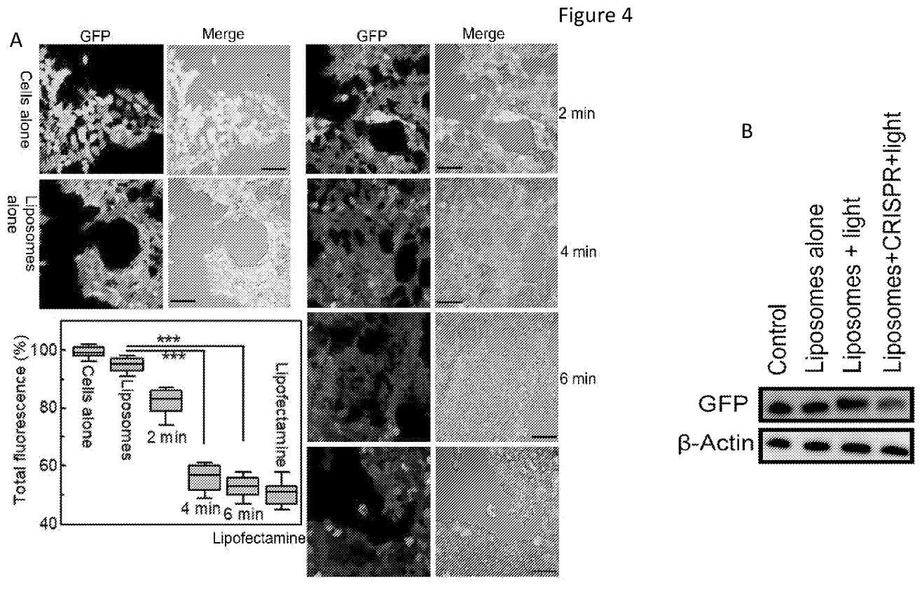

The GFP knockdown efficiency in HEK293 cells after CRISPR/Cas9 released from the light-triggered liposomes was assessed using both confocal fluorescence imaging and western blot assay (FIGS. 4A and 4B). When cells were treated with the liposomes alone, a slightly lower GFP fluorescence intensity was observed, compared with the control group without any treatment (about 5% less than the control), indicating the stability of the liposome formulation during incubation with the cells. With light illumination, CRISPR/Cas9 complex was released from the liposomes and knocked out the GFP, resulting in the clear reduction of its fluorescence signal. The lowest GFP expression level was achieved after 6 min illumination, compared with the liposome transfected cells without light irradiation (52.8% v.s. 94.8%). We also tested GFP gene knockout efficacy by employing Lipofectamine 2000 reagent as a delivery vehicle, for comparison purpose. The reduced GFP fluorescence intensity was observed in HEK293 cells at 48 hours after treatment. Although the similar GFP transfection effect was observed by using commercial lipofectamine, the on-demand gene release was achieved by using our light-triggered liposomes.

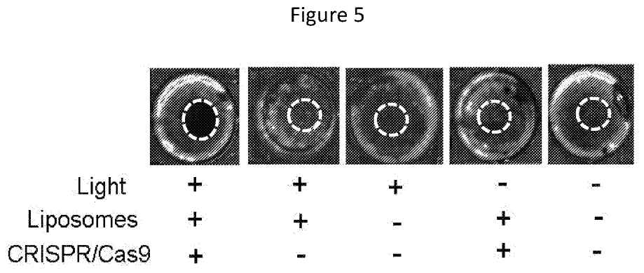

To demonstrate a unique application for spatial control of CRISPR release, we imaged the whole petri dish by using IVIS imaging system. The spatial control of CRISPR release for GFP gene knockout was demonstrated in FIG. 5. The GFP fluorescence intensity was clearly reduced within the light exposure spot, compared with the non-irradiated area of the cells in the condition of the combined treatment of light and liposome-CRISPR. We also checked the fluorescence intensity of the cells treated with the liposome-formulated CRISPR alone, light illumination alone and the combination with empty liposomes and light. These conditions did not show significant light-triggered gene knockout effect. The findings indicated that a spatially controllable way would be possible by combining a delivery vehicle with light.

Assessment of Toxicity of the Singlet Oxygen on sgRNA and Liposomes in HEK293 Cells

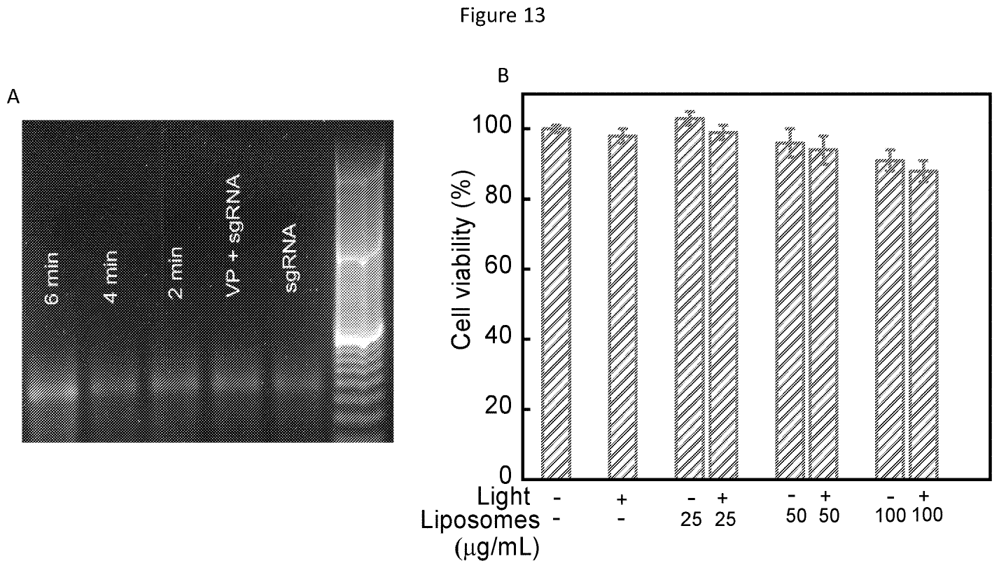

We first checked the effect of singlet oxygen on sgRNA by irradiating a mixture solution of sgRNA and VP with light illumination. As shown in FIG. 13A, there was no clear RNA damage observed compared with the control. Short lifetime of singlet oxygen prevents it from travelling larger distances, therefore it mainly causes localised, nanometre scale damage, near the photosensitizer molecule where it was generated. In this study, singlet oxygen generated from VP loaded in a lipid bilayer mainly destabilises the unsaturated lipids and consequently induces CRISPR agent release. In this way adverse effect of singlet oxygen on sgRNA will be minimised. We also assessed the toxicity of pure liposomes and combined condition with the light in HEK293 cells at 24 hr after the treatments. Compared with the control group, no significant change was observed in the viability of the cells treated with liposome concentrations up to 100 μg/mL and combination with the light illumination (FIG. 13B). These results suggested that under in vitro conditions, our liposome samples with light setting at the current study are likely not to affect the viability of HEK293 cells.

A Visual Reporter System for Rapid Quantification of Knockout Efficiency In Vivo

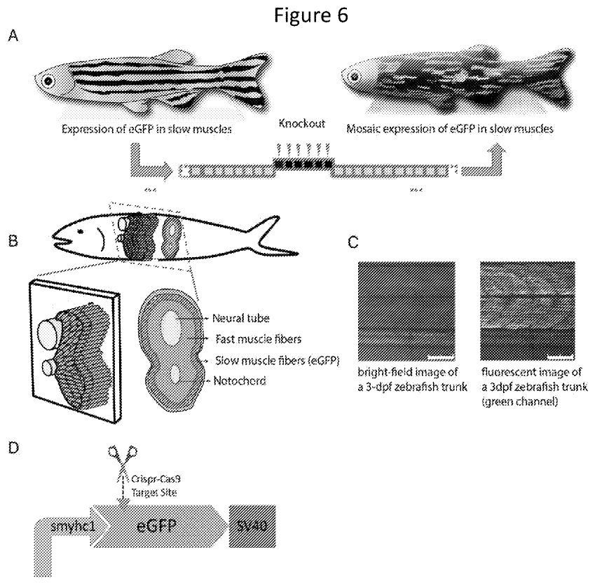

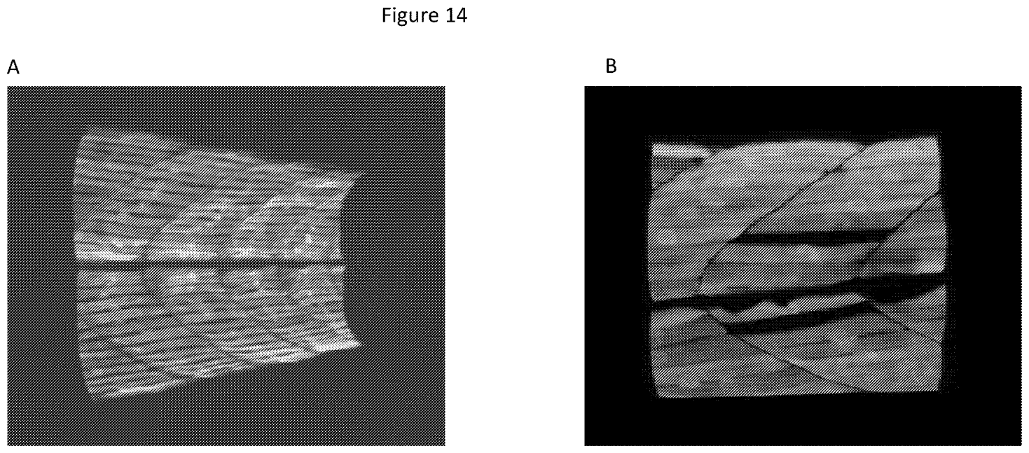

To assess the efficiency of light-sensitive liposome delivery of CRISPR/Cas9 in vivo, we developed a quantitative visual reporter system in zebrafish. We established a gene knockout strategy where eGFP expressed specifically in the slow-muscle fibers of a stable transgenic zebrafish is knocked-out by CRISPR/Cas9 (FIG. 6A). Slow-twitch muscle fibers form a single superficial layer directly under the skin arranged in parallel with the long axis of zebrafish (FIG. 6B). We used a transgenic zebrafish line (smyhc1:eGFP) where slow-muscle specific smyhc1 promoter drives the eGFP expression at slow-twitch muscle fibers (FIG. 6C). To generate a highly efficient DSB, we screened eight sgRNAs targeting eGFP using the reporter system and selected the sgRNA exhibiting highest rate (88.24%) of cutting efficiency (Table 2). To assess the efficiency of the visual reporter system, we co-injected sgRNA targeting eGFP locus with Cas9 protein into single-cell zebrafish embryos (FIG. 6D). We observed the loss of green fluorescent signal across individual slow-twitch muscle fibers showing loss of eGFP expression, whereas the control group injected without eGFP sgRNA did not exhibit any loss of green fluorescent signal (FIGS. 14 A and B). This allows rapid visual quantitation of knock-out efficiency at singe cell resolution in vivo.

TABLE 2

| | | | Cutting |
|---|---|---|---|
| | | | Efficiency |
| ID | Sequence | Target | (%) |
| sg_eGFP_01 | ATGGTGAGCAAGGGCGAGG | eGFP | 37.80 |
| sg_eGFP_02 | GACCAGGATGGGCACCACC | eGFP | 56.18 |
| | CCGG | | |
| sg_eGFP_03 | CGCCGGACACGCTGAACTT | eGFP | 6.86 |
| | GTGG | | |
| sg_eGFP_04 | CAAGTTCAGCGTGTCCGGC | eGFP | 26.33 |
| | GAGG | | |
| sg_eGFP_05 | GGCGAGGGCGATGCCACCT | eGFP | 66.04 |
| | ACGG | | |
| sg_eGFP_06 | GGGCACGGGCAGCTTGCCG | eGFP | 88.24 |
| | GTGG | | |
| sg_eGFP_07 | AGCACTGCACGCCGTAGGT | eGFP | 50.80 |
| | CAGG | | |
| sg_eGFP_08 | GCTTCATGTGGTCGGGGTA | eGFP | 49.10 |
| | GCGG | | |

Assessment on In Vivo Knockout of eGFP Gene by Light-Triggered Liposomes

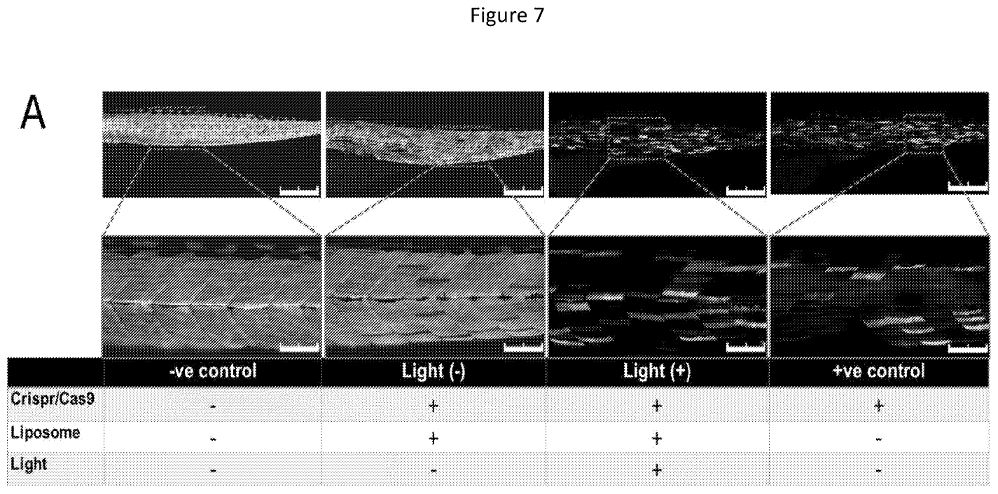

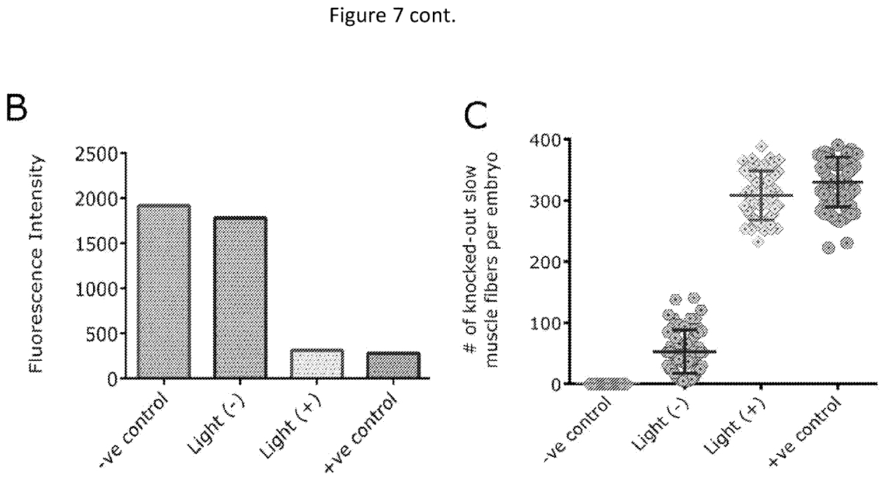

After confirmation of in vitro CRISPR transfection, we tested whether we can demonstrate targeted knockout of the eGFP by controlled release of CRISPR/Cas9 in zebrafish using light-triggered liposomes. To determine the effect of the light-triggered genome editing, we co-injected Cas9 protein and liposomes encapsulating verteporfin and eGFP sgRNA into transgenic smyhc1: eGFP zebrafish embryos. The injected embryos were randomly divided into two groups; either no light exposure or light exposure at 690 nm for 5 minutes. We used the visual reporter system described above to evaluate the efficiency of light-controlled genome editing in vivo. The initial qualitative assessment showed a major loss of green fluorescence signal in muscle fibers, suggesting light triggered release of CRISPR/Cas9 (FIG. 7A, B). The negative control group did not show any loss of green fluorescent signal, highlighting the specificity of the assay. Therefore, we proceeded to quantify the total number of slow muscle fibers knocked out in the trunk of each embryo (n=80 embryos per group; FIGS. 7C, and 14B). No light exposure resulted with modest but significant loss of green fibers compared to the negative control (FIG. 7C; −ve control, 0±0; light (−), 53.15±35.38, p<0.0001, one-way ANOVA with multiple comparison). In contrast, embryos exposed to light activation showed a dramatically significant decrease in number of green fibers compared to no light control group, implicating light-triggered knockout of eGFP in vivo (FIG. 7C; light (−), 53.15±35.38; light (+), 308.37±40.21, p<0.0001). Compared to positive control group injected with eGFP and Cas9 without liposome, embryos exposed to light activation showed a similar level of decrease in number of green slow-muscle fibers. We further observed that results from our quantitative model are consistent with the total fluorescence intensity results (FIG. 7B, C).

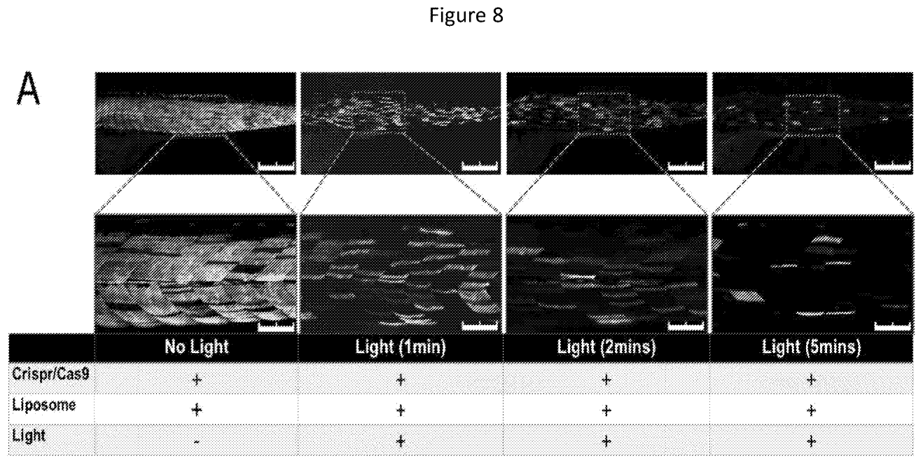

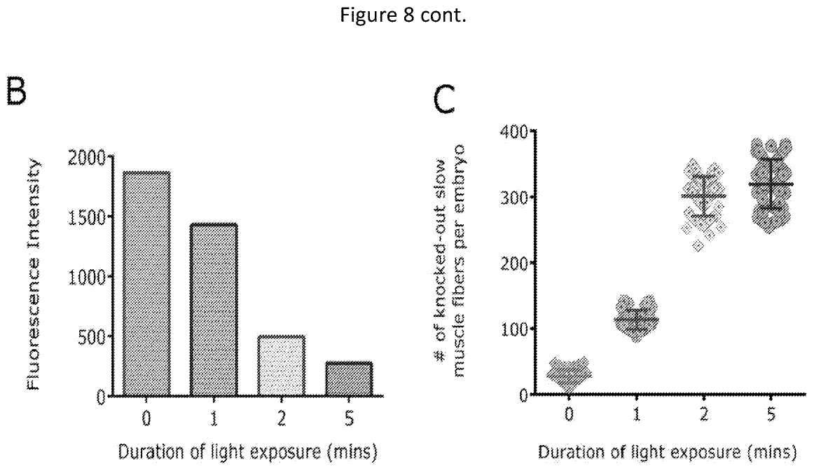

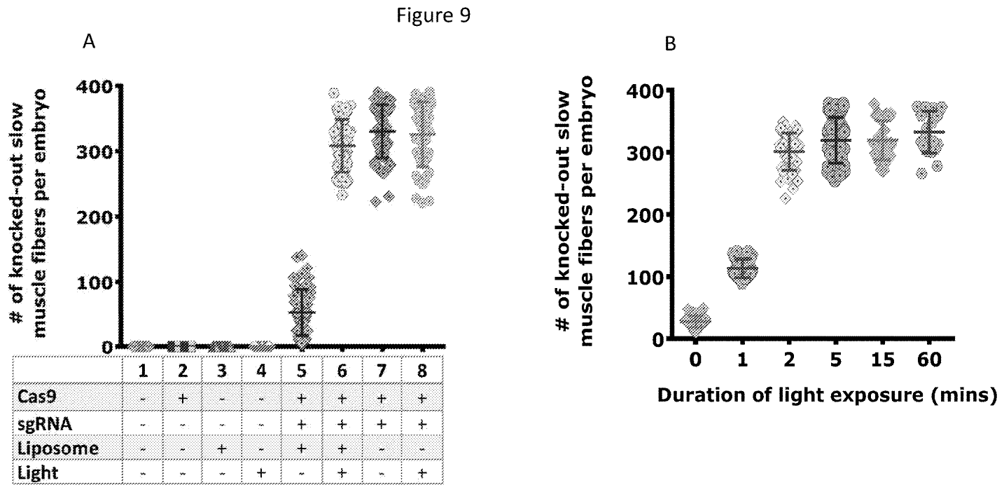

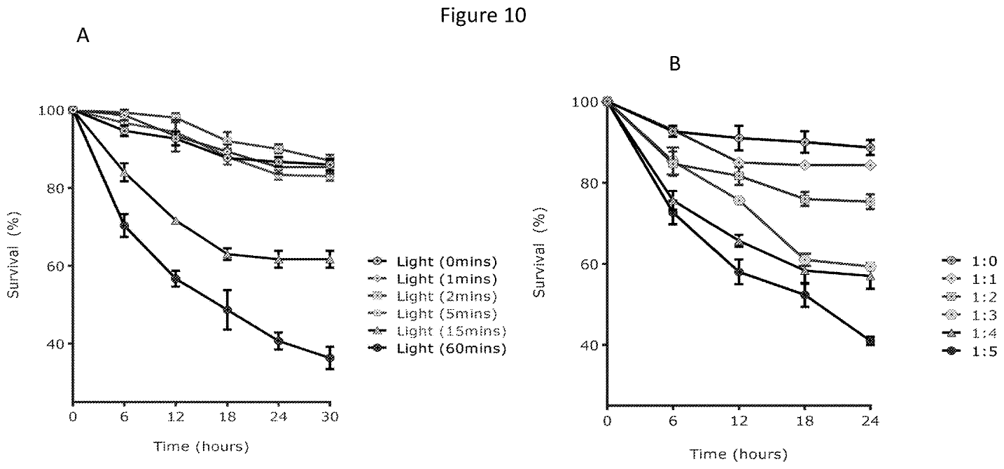

To optimize the light-triggered release of CRISPR/Cas9 in zebrafish, we first compared different light exposure times using visual reporter system as a testbed. Embryos co-injected with liposome nanoparticles and Cas9 protein were subjected to one of five different irradiation times, 1 min, 2 min, 5 min, 15 min, 60 min. Qualitative assessment implicated a difference between light exposure times, suggesting longer exposure to light leads to higher knockout rates (FIG. 8A, B). The quantitative analysis of the single-fiber analysis showed longer light exposure times leading to higher loss of green slow-muscle fibers. (FIG. 8C; No Light, 27.84±9.81; Light (1 min), 113.12±14.77, Light (2 min), 300.67±30.16 Light (5 min), 326.12±36.55; n=60 embryos per group). However, we did not observe any significant difference in loss of green fluorescent signal at light illumination longer than 5 minutes (FIG. 9B; Light (5 min), 326.12±36.55; Light (60 min), 332.65±33.60, p=0.43). Light illumination up to 5 min did not affect the embryo survival, however longer exposure to red light led to reduced embryo viability. At 60 min light illumination, 36% of zebrafish morphologically normal looking zebrafish embryos remained alive (FIG. 10A).

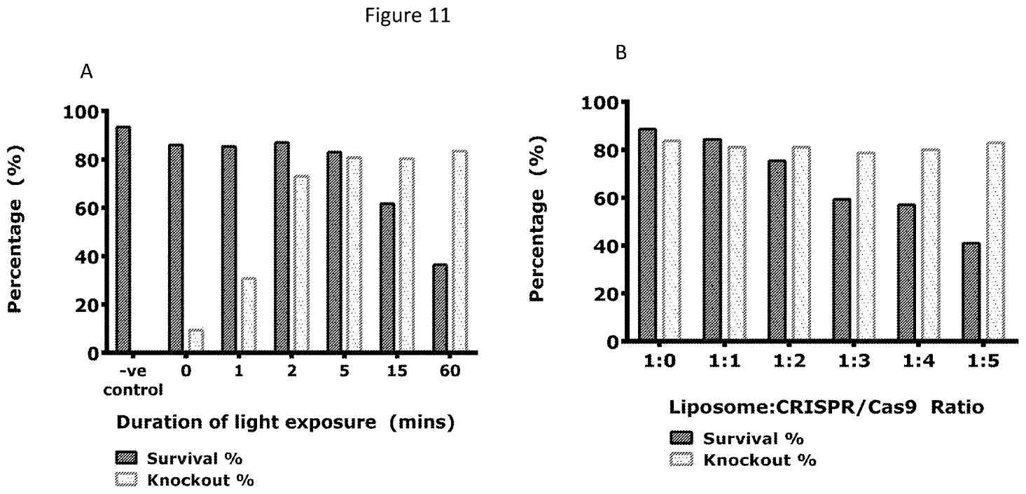

Next, we investigated the effect of liposome nanoparticles concentration on light-triggered release of CRISPR sgRNA. We also determined the effect of liposome concentration on embryo toxicity by measuring the hatching rate of zebrafish embryos injected with different liposome concentrations. While the higher concentration of liposome led to increased mortality in zebrafish embryos (FIG. 10B), efficiency of light-triggered release of CRISPR remained unaffected (FIG. 11B).

### Discussion and Conclusions

The ability to manipulate any genomic sequence by CRISPR gene editing has created diverse opportunities for biological research and medical applications. However, further advancement of gene editing requires the development of optimal delivery vehicles. Non-viral delivery is particularly advantageous, as it avoids insertional errors and it allows tight control over the dose, duration, and specificity of delivery. The liposomal platform investigated here is able to simultaneously release controlled amounts of the Cas9 nuclease and matching amounts of gRNA in a way that is spatially and temporally controlled by an external light beam applying safe levels of 690 nm light (0.15 mW/cm²) to tissue surface. While light required to trigger our liposomes penetrates tissue only up to a few millimeters, optical fibre approaches developed for photodynamic therapy of cancer make it possible for these liposomes to be applied in deep tissue as well.

Light-triggered liposomal release of CRISPR reagents offers previously unavailable option for gene editing to be localised in space and time; such four-dimensional control will be important for novel research applications and for further clinical translation of the CRISR-Cas9 technique.

Lipid nanoparticles and conventional liposome-based delivery used for CRISPR transfection in preclinical settings suffer from a drawback. After internalization of the through the endocytic pathway, most of these carriers become entrapped in endo/lysosomes where the enzymatic degradation may result in deactivation of CRISPR components before they are able to be released to perform their gene editing action. Therefore ensuring rapid endo/lysosomal escape of the cargos is required for efficient CIRSPR/Cas9 transfection via lipid-based nanoparticles. Our light-triggerable liposomes overcome the issue of endo/lysosomal entrapment, because, as we established earlier, VP activated by light illumination generates sufficient singlet oxygen to destabilise not only the liposomes but also the liposomal and endo/lysosomal membranes.

The ability of our liposomes to deliver defined amounts of intact Cas9 represents a key advantage of this formulation for efficient and nontoxic gene editing. The Cas9 protein is large (˜160 kDa) and this prevents its direct delivery to cells (Glass et al., Trends in Biotechnology, 2018, 36(2): p. 173-185). We found that our liposome encapsulation enables direct Cas9 protein delivery to cells and may partially protect it from degradation. Such direct nuclease delivery in CRISPR offers the immediate function without protein expression process and the most rapid therapeutic activity as there is no cellular translation or transcription. Direct delivery of purified nuclease proteins or Cas9 protein-gRNA complexes is additionally important because it yields high levels of gene editing [56]. This is consistent with the results reported here of high efficiency of the eGFP knockout observed in HEK293 cells (up to 52%) and in zebrafish embryo (up to 77%) treated with light-triggered liposomes compared to the control group (FIGS. 4, 5 and 7). Our result confirms that light-triggered CRISPR/Cas9 release does not compromise the genome editing activity in the target loci. Transient protein delivery via liposomes also restricts the duration of nuclease activity potentially reducing off-target editing as the nuclease has less opportunity for promiscuous action. Our approach may therefore play an important role in ensuring precision and safety of the CRISPR-Cas9 tools. The liposomes also enable direct gRNA delivery to cells which is not straightforward because the long phosphate backbone of gRNA is too negatively charged to passively cross the membrane. Furthermore, the liposomes may help the gRNA to avoid nuclease degradation. We found in this work that our liposome encapsulation provides sufficient protection for CRISPR reagents to gain cellular entry in HEK293 cells and in zebrafish embryos and subsequently escape from the endosomes to enter the cytoplasm while remaining functional. We also observed only a modest leakage of liposomal contents in controls which were not exposed to light. This is tentatively explained that the cell contents may compromise the integrity of liposomal membranes. The liposomal nanoparticles demonstrated minimal cytotoxicity both in HEK293 cells and zebrafish embryos, under the current experimental conditions (FIG. 13).

The data shown in FIG. 4 compare our light-triggered liposomal delivery with CRISPR delivered using Lipofectamine, a commercially available liposome delivery vehicle for nucleic acids and gene editing proteins. Lipofectamine draws on the ability of lipids to spontaneously form nanoparticles in aqueous solution in order to protect their hydrophobic tails from the solvent. By simple mixing, a payload may be encapsulated within a lipid nanoparticle. Lipofectamine contains cationic lipids that complex with the negatively charged nucleic acid molecules and this reduces the effect of electrostatic repulsion of the negatively charged cell membrane. This additionally protects nucleic acids from nucleases and allows them to be taken up by target cells. Lipofectamine has been previously used in conjunction with the CRISPR system for various application purposes, including generation of an immunodeficiency model (Horii, T., et al., Generation of an ICF syndrome model by efficient genome editing of human induced pluripotent stem cells using the CRISPR system. 2013. Int. J. Mol. Sci. 14(10): p. 19774-19781), multiplex genome editing (Sakuma, T., et al., Multiplex genome engineering in human cells using all-in-one CRISPR/Cas9 vector system. 2014. Sci Rep 4: p. 5400), and gene therapy of cystic fibrosis and bladder cancer (Schwank, G., et al., Functional repair of CFTR by CRISPR/Cas9 in intestinal stem cell organoids of cystic fibrosis patients. 2013. Cell Stem Cell 13(6): p. 653-658; Liu, Y., et al., Synthesizing AND gate genetic circuits based on CRISPR-Cas9 for identification of bladder cancer cells. 2014. Nature Communications 5: p. 5393). The in vitro CRISPR transfection efficiency via our light-triggered liposomes and Lipofectamine was found to be comparable (52% v.s. 50% GFP level reduction in FIG. 4). However, unlike Lipofectamine, our liposomes can be triggered by light allowing spatial and temporal control of gene editing, moreover they are feasible to be functionalized with different ligands of interests

The light-triggerable CRISPR delivery vehicles reported here are biocompatible and made entirely from clinically-approved components using a simple synthesis method. This design avoids the need for numerous manufacturing steps in the future scaling-up process. It is also important from a commercial and regulatory point of view that the entire gene therapy product can be packaged in a single vehicle. In vivo gene editing benefits from tissue-specific targeting (e.g. using tissue specific promoters of Cas9) to prevent undesirable off-target gene editing events. Targeted delivery of liposomes is well established, and such molecular targeting is also directly applicable to the CRISPR-carrying liposomes investigated here. Liposomes are also well suited to co-delivery of multiple components, and this is highly relevant as novel CRISPR refinements may require simultaneous delivery of multiple functional entities. The liposomes are entirely DNA-free and this will help avoid DNA toxicity and stimulating immune responses. Favourable biodistribution in specific disease conditions may be achieved by optimising formulations and by a suitable route of administration.

### Example 2—Spatial and Temporal Control of CRISPR/Cas Gene Editing Via a X-Ray-Triggered Liposome System

Light triggering modality has limited tissue penetration depth (few mm) when applying light-triggered liposomes to the deep tumour treatment. As a result of this modest penetration depth, visible light may not be able to activate photosensitizers located deeply in the body and generate sufficient amount of singlet oxygen (¹O₂) or other reactive oxygen species (ROS) to release the liposome cargo required for the therapeutic effects. With its excellent tissue penetration depth, X-ray radiation for liposome triggering offers an alternative approach to yield both spatial targeting (such as to a tumour site) via standard radiotherapy approaches such as the Gamma-knife (Begg, A. C., et al., Nature Reviews Cancer, 2011. 11(4): p. 239-253) and triggered release of encapsulated contents from the liposomes once they are located at the target site. Importantly, the X-ray liposome triggering can be used concurrently with radiation therapy, a common treatment modality in cancer.

We previous showed that verteporfin can be sufficiently activated by low dose X-ray radiation (2-4 Gy doses) to trigger drug release from instability in the membrane of verteporfin-containing liposomes (Deng, W., et al., Controlled gene and drug release from a liposomal delivery platform triggered by X-ray radiation. 2018. Nature Communications, 9(1): p. 2713). We demonstrated the in vivo therapeutic effect in a BALB/c nu/nu mouse model bearing a xenograft mode of cancer by using X-ray triggered liposomes loaded with verteporfin, gold nanoparticles and a chemotherapy drug doxorubicin (Dox).

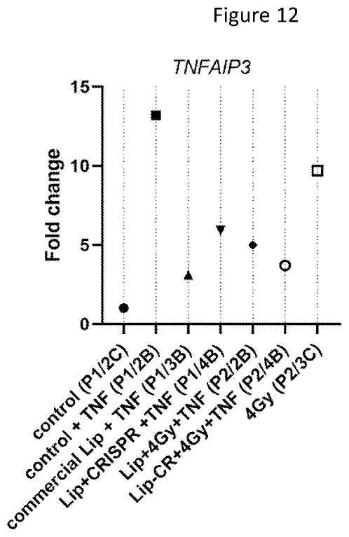

We loaded CRISPR/Cas9 into the X-ray triggered liposomes loaded with verteporin, gold nanoparticles and CRISPR/Cas9 targeting the TNFAIP3 gene, and assess its capability on A20 knockout in human embryonic kidney cells (HEK293). Liposomes loaded with verteporin and gold were prepared as described in Deng et al. (2018) Nature Communications, vol. 9, Article number 2713. A20, encoded by the TNFAIP3 gene, promotes microbial tolerance as a negative regulator of NF-κB signaling: an evolutionarily ancient and central pathway for activating innate and adaptive immune responses. Our results on A20 knockout by using X-ray triggered CRISPR-liposome are shown in FIG. 12. After these treatments as indicated, TNFAIP3 gene expression level (relative to GAPDH) was changed to the different levels. Compared with the positive control (commercial liposome+CRISPR, green rectangle), gene expression was also clearly reduced after X-ray triggered liposome loaded with CRISPR (purple rectangle). We also checked other the treatment conditions, including empty liposomes alone, liposomes with CRISPR alone, X-ray alone, these conditions did not affect the gene knockout efficiently. These result indicate that liposomes can be disrupted by X-ray radiation to release CRISPR/Cas9 into target cells in deep tissue.

1. A liposomal nanoparticle comprising:
(a) a liposomal vehicle comprising:
(i) one or more liposome forming lipids; and
(ii) one or more destabilising agents capable of forming reactive oxygen species when exposed to an inducer; and
(b) a genome editing agent, or part thereof.

2. The liposomal nanoparticle of claim 1, wherein the genome editing agent or part thereof is a CRISPR complex or part thereof.

3. The liposomal nanoparticle of claim 1, wherein the liposomal vehicle further comprises cholesterol.

4. The liposomal nanoparticle of claim 1, wherein the one or more liposome forming lipids are phospoholipids.

5. The liposomal nanoparticle of claim 4, wherein the phospoholipids are DOPC and DOTAP.

6. The liposomal nanoparticle of claim 1, wherein the one or more destabilising agents is a photosensitiser.

7. The liposomal nanoparticle of claim 1, wherein the one or more destabilising agents is verteporfin.

8. The liposomal nanoparticle of claim 1, wherein the one or more destabilising agents is a metal nanoparticle.

9. The liposomal nanoparticle of claim 1, wherein the metal nanoparticle is a gold nanoparticle.

10. The liposomal nanoparticle of claim 1, wherein the one or more destabilising agents is a photosensitiser and a metal nanoparticle.

11. The liposomal nanoparticle of claim 1, wherein the one or more destabilising agents is verteporfin and gold nanoparticles.

12. The liposomal nanoparticle of claim 1, wherein the inducer is electromagnetic radiation.

13. The liposomal nanoparticle of claim 12, wherein the inducer is light.

14. The liposomal nanoparticle of claim 1, wherein the inducer is high energy electromagnetic radiation.

15. The liposomal nanoparticle of claim 1, wherein the inducer is X-ray or gamma-radiation.

16. The liposomal nanoparticle of claim 1, wherein the genome editing agent, or part thereof comprises a guide RNA of a CRISPR complex that specifically binds to a target DNA.

17. The liposomal nanoparticle of claim 1, wherein the genome editing agent, or part thereof comprises a CRISPR-associated protein or an RNA encoding a CRISPR-associated protein.

18. The liposomal nanoparticle of claim 1, wherein the genome editing agent, or part thereof comprises a CRISPR-associated protein or an RNA encoding a CRISPR-associated protein, and a guide RNA that specifically binds to a target DNA.

19. The liposomal nanoparticle of claim 17 or 18, wherein the CRISPR-associated protein is cas9, or a variant thereof.

20. The liposomal nanoparticle of claim 1, wherein the genome editing agent further comprises a cationic polymer.

21. A composition comprising the liposomal nanoparticle of any one of claims 1 to 20.

22. The composition of claim 21, further comprising a pharmaceutical carrier.

23. A liposomal system for delivery of a CRISPR complex or part thereof, comprising:
(a) a liposomal vehicle comprising:
(i) one or more liposome forming lipids; and
(ii) one or more destabilising agents capable of forming reactive oxygen species when exposed to an inducer; and
(b) a genome editing agent or part thereof;
wherein the genome editing agent is released from the liposome by exposure of the destabilising agent to the inducer.

24. The liposomal system of claim 23, wherein the genome editing agent is a CRISPR complex or part thereof.

25. The liposomal system of claim 23, wherein the inducer is electromagnetic radiation.

26. The liposomal system of claim 23, wherein the inducer is light.

27. The liposomal system of claim 23, wherein the inducer is high energy electromagnetic radiation.

28. The liposomal system of claim 23, wherein the inducer is X-ray or gamma-radiation.

29. A method of modifying a genome of a cell, comprising administering the liposomal nanoparticle of any one of claims 1 to 20, or the composition of any one of claim 21 or 22, to a cell, and exposing the liposomes to an inducer to thereby destabilise the liposome and release the genome editing agent.

30. The method according to claim 29, wherein the cell is in a subject.

31. The method of claim 29, wherein the inducer is electromagnetic radiation.

32. The liposomal system of claim 29, wherein the inducer is light.

33. The liposomal system of claim 29, wherein the inducer is high energy electromagnetic radiation.

34. The liposomal system of claim 29, wherein the inducer is X-ray or gamma-radiation.

35. The method of claim 29, wherein genome editing agent is a CRISPR complex or part thereof.

36. A method of preparing a liposomal nanoparticle, comprising combining one or more liposome forming lipids, one or more destabilising agents capable of forming reactive oxygen species when exposed to an inducer, and a genome editing agent, under conditions which promote formation of a liposomal vehicle encapsulating the genome editing agent.

37. The method of claim 36, further comprising combining cholesterol.

38. The method of claim 36, wherein the one or more amphipathic vesicle forming lipids are phospoholipids.

39. The method of claim 38, wherein the phospoholipids are DOPCC and DOTAP.

40. The method of claim 36, wherein the one or more destabilisers is a photosensitiser.

41. The method of claim 36, wherein the photosensitiser is verteporfin.

42. The method of claim 36, wherein the one or more destabilisers is gold nanoparticle.

43. The method of claim 36, wherein the one or more destabilisers is verteporfin and gold nanoparticles.

44. The method of claim 36, wherein the genome editing agent is a CRISPR complex or part thereof.

45. The method of claim 44, wherein the genome editing agent comprises a CRISPR-associated protein or an RNA encoding a CRISPR-associated protein, and a guide RNA that specifically binds to a target DNA.

46. The method of claim 45, wherein the CRISPR-associated protein is cas9, or a variant thereof.

47. The method of claim 45 or 46, wherein the CRISPR complex further comprises a cationic polymer.

48. A kit for preparing a liposomal nanoparticle claim 1, comprising:
(i) one or more liposome forming lipids;
(ii) one or more destabilising agents capable of forming reactive oxygen species when exposed to an inducer; and
(iii) a genome editing agent.


SEQUENCE LISTING (1 sequence)

SEQ ID NO: 1 - protein, 1368 aa, Streptococcus pyogenes [sequence omitted]